(19) United States
(12) Patent Application Publication
(10) Pub. No.: US 2007/0054388 A1
(43) Pub. Date: Mar. 8, 2007

(54) Method for immobilising microorganisms, related material, and use thereof

(76) Inventors: Maria De Fatima Teixeira Cardoso Da Silva, Canelas (PT); Pierre Marie Jean Strehaiano, Escalquens (FR); Rui Chedas Sampaio, Parede (PT)

Correspondence Address: WENDEROTH, LIND & PONACK, L.L.P., 2033 K STREET N. W., SUITE 800, WASHINGTON, DC, 20006-1021, US

(21) Appl. No.: 10/552,457

(22) PCT Filed: Apr. 9, 2003
(86) PCT No.: PCT/IB03/01753

§ 371 Date: Sep. 20, 2006

(51) Int. Cl. C12P 7/06; C12N 1/18

(52) U.S. Cl. 435/161; 435/254.21

(57) ABSTRACT

The present invention relates to a method for immobilising microorganisms (particularly yeasts or bacteria), a related material, and the use thereof in fermentation or bioconversion. Specifically, the present invention relates to the immobilisation of microorganisms in optionally dehydrated polymeric spheres consisting of two or more layers. The technical problem is that only part of the total microorganism population, namely the microorganisms adjacent to the edge of the sphere, is actually active in conventional spheres. According to the present invention, this problem is solved by providing a nutrient supply in the center of the sphere and placing the microorganisms in a layer adjacent thereto, the resulting combination optionally being coated with a sterile layer defining, in this embodiment, a triple-layer sphere.

### FIELD OF APPLICATION OF THE INVENTION

The present invention proposes a novel and innovative method for immobilizing microorganisms (in particular, yeasts or bacteria), the respective product and its use in fermentation and bioconversion methods.

In particular, this invention concerns the immobilizing of microorganisms in polymer spheres formed by two or more layers with these spheres capable of being dehydrated.

These spheres may be used in developing fermented beverages or in conducting bioconversion reactions. Bioconversion reactions are transformations caused by the microorganisms, but which do not require growth of these same microorganisms. Applications particularly aim at the field of fermented beverages such as, in particular, still wines, beer, sparkling wines, soft drinks, mead and alcohol (ethyl).

### TECHNOLOGICAL BACKGROUND

It is well known that there are various ways of immobilizing microorganisms:
Homogeneous spheres, also called, “monolayer” or “single layer” spheres. These are comprised of a gel containing the microorganisms, as described in the patent documents FR2320349 and FR2359202. It is known that these spheres do not prevent the release of the microorganisms (called seeping out) towards the reaction medium, which in some cases is not compatible with the requirements of the method (sparkling wines for example, because of the undesirable cloudiness that this seeping-out causes).
EP0173915 describes the formation of a sterile layer around the core of the gel which contains the microorganisms, this layer being implemented to avoid this seeping-out phenomenon, and in sparkling wines the patent develops the application of spheres or threads obtained and called “double layer”. This type of product may be wet or partially dehydrated as explained in patent EP0350374.

The problem that is currently posed is that, as is known and demonstrated in spheres having such a structure (monolayer or double layer), only a small fraction of the total population of microorganisms are truly in activity. In fact, the nutrient substrates are consumed by the microorganisms located in the periphery of the layer. On the other hand, for some cases, the medium does not contain all the nutrient elements necessary for good activity of these immobilized microorganisms. Evidently, this problem is common to biocatalysts configured in other forms, such as those presented in the form of threads as described in said document EP0173915. In the case of biocatalysts where the microorganisms are immobilized, those that are found at the periphery or in close proximity to it (when there is an external protective layer without microorganisms) consume most of the nutrient elements that come from outside, making access impossible or decreasing access to microorganisms located near the interior, which means that these microorganisms remain relatively inactive.

### DESCRIPTION OF THE INVENTION

In spite of the fact that the description that follows may be based on the case of a product with immobilized microorganisms, hereafter called biocatalyst, in the form of spheres, this form being used by way of example, the present invention may be implemented in any form whatsoever, threads or plates, whatever may be compatible with the general principles stated and with the field of application defined in the claims.

According to the present invention, the problem stated previously is resolved by creating a nutrient supply in the center of the sphere and by placing microorganisms in a layer adjacent to the latter, the entire combination optionally being followed by covering with a sterile layer, which in this case defines a sphere with three layers.

As indicated above for the biocatalyst, while the following description with the two or three layers is used by way of example, in general the preferred embodiment, it can be understood as forming a multilayer biocatalyst with more alternating layers for example, layers of nutrient supplies alternating with layers that contain microorganisms according to the general principle of the present invention, the entire combination optionally being surrounded by a sterile layer. Although the layers should preferably be concentric, this is not essential.

In this way, the nutrient supply may be configured as a group of distinct sites surrounded by the layer that includes the microorganisms and the entire combination may be surrounded by a sterile layer.

According to a preferred embodiment of the invention, when there is a sterile external layer, it should be free of microorganisms and should not be permeable to microorganisms.

According to a preferred embodiment, the aim of this invention is a double or triple layer sphere with the following characteristics:
1. A nutrient supply for supplying the immobilized microorganisms in the adjacent layer with nutrient elements which ensure their good activity, however, the medium into which the spheres will be introduced is not capable of supplying a sufficient quantity. These nutrient elements may be slightly different in nature and in concentration according to the legislation in force in the country where it is being used; however, in general, they supply a source of nitrogen (ammoniacal, amino, according to the microorganism), mineral salts (phosphates, sulfates, potassium, magnesium etc.), oligoelements (iron, copper, zinc, etc.) and vitamins (thiamine, biotin etc.). The extracts of autolyzed yeasts will advantageously be used as a nutrient source.
   According to the type of use, this nutrient supply may also contain a carbonaceous substrate such as a fermenting sugar or even a complete natural medium such as a grape must which may or may not be diluted.
   According to this invention, this nutrient solution is mixed with a polymer capable of being transformed into a gel, for example, sodium alginate. An adequate concentration of this polymer is between 1% and 3% in aqueous solution.
2. A layer adjoining the nutrient supply comprised of a solution capable of being transformed into a gel (hereafter called fixation medium) and a population of microorganisms in suspension in this solution. The microorganisms may in particular be yeasts or bacteria. For the application of type ‘in-bottle fermentation’ of sparkling wines, or resumption of fermentation of musts after fermentation has stopped, it is advisable to use selected strains of the yeasts Saccharomyces cerevisiae and Saccharomyces uvarum.
   Apart from Saccharomyces, other genera of yeasts may be used for example in the production of beer or beverages with low alcohol content or even in bioconversion reactions. For example, the genus Schizosaccharomyces will be used more for deacidification of acidic musts, the bacteria Oenococcus oeni or Lactobacillus for malolactic fermentation and the genus Candida for the bioconversion of xylose to xylitol.
   According to another characteristic of the invention, the concentration of the polymer varies between 1% and 50%.
   The microorganisms may stem from a fresh culture made according to the standards in force in the industry or even from an active or lyophilized dry commercial preparation and used according to the advice of the manufacturer.
   An additional external layer (in the case of spheres with 3 layers) which is prepared from a type of polymer identical to the other two layers and capable of being transformed into a gel. The concentration of the polymer is identical to that used for the other layers. According to an additional characteristic of the invention, in certain applications an enzymatic preparation or a preparation of organic components could be introduced into the external layer:
      For example (i), a lysozyme solution added to the external layer to prevent the growth of undesirable bacteria which are sensitive to this enzyme (known example of lactic bacteria in winemaking) and which could be at the source of a cloudiness that is difficult to remove in the case of sparkling wines.
      For example (ii), the walls of yeasts that will fix fatty acids which are inhibitors of the activity of the fixed microorganisms, in case a treatment is employed for stopping fermentation.
   According to another characteristic of the invention, the particles thus formed, called triple-layer spheres, are produced in a single step.
   The simultaneous implementation of three concentric layers may be accomplished by resorting to a device with concentric tubes which define two concentric annular zones around a central zone which is also concentric, incorporating the nutrient supply by the central tube, the incorporation of microorganisms and fixation medium through the annular zone defined by the external portion of the central tube and by the internal portion of the intermediate tube and by incorporating the external layer through the annular zone defined by the external portion of the intermediate tube and by the internal portion of the external tube.
   The diameter of the wet spheres is between 1 and 5 mm. Gelation is carried out by passing a crosslinking agent, in particular calcium chloride, into a solution according to a known method and preferably where the crosslinking of the sphere takes place from the exterior to the interior. According to another characteristic of the invention, these spheres may be more or less dehydrated to a final water activity (AW) of 0.1 to 0.5, preferably 0.3 to 0.4. This dehydration is carried out with a drying technique, by fluidized bed or the use of an oven. These spheres are stored in a package resistant to vapor and to air and preferably kept at a relatively low temperature, the ideal temperature being 4° C. Under these conditions, it is possible to store the product for several months.

The examples that follow illustrate some of the applications, characteristics and advantages of the invention.

### EXAMPLE 1:

Preparation of Spheres with Triple Layer of Alginate, Comprising Immobilized Yeasts, Saccharomyces Cerevisiae, and Prepared Beforehand to Resist a Medium Rich in Alcohol

The spheres are prepared from a solution of sodium alginate, an unbranched polymer extracted from algae and composed of α-D-manuronic acid and β-D-guluronic acid. The yeasts, which may or may not be acclimated to the alcohol beforehand, are mixed with one part of this solution in a tank. For this example, a strain of Saccharomyces cerevisiae EC1118 sold under the Lalvin brand will be used.

Next and as a result of a device with concentric tubes, this solution passes through a vibration system which enables the formation of the spheres.

During contact with a 0.2M solution of calcium chloride, they are immediately gelated, the contact time being one-half hour.

The spheres so formed are washed by immersion for 10 minutes in demineralized water.

The spheres are subsequently partially dehydrated in a fluidized bed. This drying makes it possible to obtain a water activity (AW) between 0.3 and 0.4. The drying temperature is less than or equal to 40 C. The diameter of the spheres obtained is from 1.5 to 4 mm. After quality control, the immobilized and dehydrated yeasts are packaged and stored at 4° C. before use.

### EXAMPLE 2:

Stability of Yeasts During Storage

In order to confirm the preservation of the activity of the spheres with the strain of Saccharomyces cerevisiae following storage for 6 months at 4° C., testing is carried out in the following way:
30 g of spheres prepared according to Example 1, covered with a sucrose solution at 50 gL⁻¹ (volume 100 mL) at 37° C.
The solution is kept at 37° C. and the change over time of the concentration of sugar is to be followed.
After two hours, the concentration of the sugar is less than 2 gL⁻¹, which corresponds to the end of fermentation.

This reference control is carried out at time 0 (immediately after the production of the spheres) and also after 6 months of storage at 4° C. The end of fermentation occurs after the same fermentation time of 2 hours. Therefore, the immobilized yeasts prepared according to Example 1 appear to be stable for 6 months of storage at 4° C.

1. Method for immobilizing microorganisms characterized in that an adequate nutrient supply for said microorganisms is incorporated into a medium for the fixation of the microorganisms and crosslinking of this support medium is carried out.

2. Method as claimed in claim 1, characterized in that the nutrient supply is placed in the zone furthest from the external edge of the fixation medium of the microorganisms.

3. Method as claimed in claim 1 characterized in that the nutrient supply is physically separated into distinct zones, being located in the fixation medium of the microorganisms with a greater density in the zones furthest from the external edge of said medium.

4. Method as claimed in claim 1 characterized in that the nutrient supply includes a source of nitrogen (ammoniacal, amino, according to the microorganism), mineral salts ( phosphates, sulfates, potassium, magnesium, among others), oligoelements (iron, copper, zinc, among other) and vitamins (thiamine, biotin, among others).

5. Method as claimed in claim 1 characterized in that complex nutrient sources are utilized, such as extracts of autolyzed yeasts.

6. Method as claimed in claim 1 characterized in that the nutrient supply comprises a carbonaceous substrate, such as fermentable sugar or even a complete culture medium such as a must which may or may not be diluted.

7. Method as claimed in claim 1 characterized in that the nutrient supply is mixed with a polymer, in particular sodium alginate, capable of being transformed into a gel.

8. Method as claimed in claim 7 characterized in that the solution capable of being transformed into a gel has a polymer concentration varying between 1% and 3%.

9. Method as claimed in claim 1 characterized in that the layer(s) adjoining the nutrient supply is (are) formed from a solution capable of being transformed into a gel and microorganisms in suspension in this solution.

10. Method as claimed in claim 9 characterized in that the solution capable of being transformed into a gel has a polymer concentration varying between 1% and 50%.

11. Method as claimed in claim 9 characterized in that the microorganisms are yeasts or bacteria.

12. Method as claimed in claim 11 characterized in that strains of yeasts selected from Saccharomyces cerevisiae or Saccharomyces uvarum are utilized during bottle fermentation of sparkling wines or upon resumption of fermentation of musts displaying a slowing-down or stopping of alcoholic fermentation.

13. Method as claimed in claim 11 characterized in that yeasts from the genus Schizosaccharomyces are used during deacidification of acidic musts.

14. Method as claimed in claim 11 characterized in that the bacteria Oenococcus oeni or Lactobacillus are utilized during malolactic fermentation.

15. Method as claimed in claim 11 characterized in that yeasts from the genus Candida are utilized during bioconversion of xylose into xylitol.

16. Method as claimed in claim 1 characterized in that a sterile external layer without microorganisms and lacking permeability to the microorganisms existing in the fixation medium, is added to the fixation medium of the microorganisms simultaneously or after the crosslinking step of the latter.

17. Method as claimed in claim 16 characterized in that the external layer is comprised of a polymer which is identical in nature to the interior layers and capable of being transformed into a gel and with a concentration identical to that of the other layers.

18. Method as claimed in claim 16 characterized in that an enzymatic preparation or a preparation of organic compounds is introduced into the external layer.

19. Method as claimed in claim 18 characterized in that a lysozyme solution is added to said external layer, preventing the growth of undesirable species sensitive to this enzyme, especially lactic bacteria in winemaking.

20. Method as claimed in claim 18 characterized in that walls of yeasts are added to said external layer which will fix the fatty acids which are inhibitors of the activity of the fixed microorganisms during treatment to stop fermentation.

21. Product with immobilized microorganisms characterized in that it includes a nutrient supply incorporated in the fixation medium of the microorganisms.

22. Product as claimed in claim 21 characterized in that it comprises three layers, the internal layer being formed by the nutrient supply, the intermediate layer being formed by the microorganisms and the fixation medium, and with the sterile external layer being without microorganisms and impermeable to the microorganisms listed.

23. Method as claimed in claim 1 for the production of a product characterized in that it comprises three layers, the internal layer being formed by the nutrient supply, the intermediate layer being formed by the microorganisms and the fixation medium, and with the sterile external layer being without microorganisms and impermeable to the microorganisms listed, wherein the product is implemented in a single step with three layers by using concentric tubes which define two concentric annular zones around a central zone which is also concentric, and by carrying out the incorporation of the nutrient supply through the interior of the central tube, the incorporation of microorganisms and of the respective fixation medium through the annular zone defined by the external portion of the central tube and by the internal portion of the intermediate tube and by incorporating the external layer through the annular zone defined by the external portion of the intermediate tube and by the internal portion of the external tube.

24. Method as claimed claim 23 characterized in that the crosslinking of the product, dispensed by the system of tubes, is carried out by passing the latter through a solution of a crosslinking agent.

25. Method as claimed in claim 24 characterized in that the polymer capable of being transformed into a gel is identical in nature in the three layers of the product, and in the case the latter is sodium alginate, the crosslinking agent is calcium chloride, thus by carrying out crosslinking of said product exclusively from the exterior towards the interior.

26. Method as claimed in claim 23 characterized in that the product dispensed by the device of concentric tubes is cut by a vibration device, thus forming spheres.

27. Method as claimed in any claim 23 characterized in that the product subsequently undergoes partial dehydration to a final AW of 0.1 to 0.5, preferably 0.3 to 0.4, in particular by using a drying technique with fluidized bed or the use of ovens.

28. Product as claimed in claim 22, produced according to the method of claim 26 characterized in that it has a spherical shape with three layers.

29. Product as claimed in claim 28 characterized in that the external diameter of the wet spheres is between 1 mm and 5 mm.

30. Use characterized by the utilization of the product of claim 21 for the fermentation of beverages in the bottle.

31. Use characterized by the utilization of the product of claim 21 for the resumption of fermentation of musts displaying a slowing-down or stopping of the alcoholic fermentation.
